(19) United States
(12) Patent Application Publication
(10) Pub. No.: US 2016/0287615 A1
(43) Pub. Date: Oct. 6, 2016

(54) NOVEL ENCAPSULATION OF FLUORESCENT, PHOTO-SENSITIVE, OR OXYGEN-SENSITIVE ACTIVE INGREDIENT FOR TOPICAL APPLICATION

(71) Applicant: BioPharmX, Inc., Menlo Park, CA (US)

(72) Inventors: Kin F. Chan, Los Gatos, CA (US); Anja B. Krammer, Menlo Park, CA (US); Douglas W. Thomas, Palo Alto, CA (US)

(73) Assignee: BioPharmX, Inc., Menlo Park, CA (US)

(21) Appl. No.: 15/090,595

(22) Filed: Apr. 4, 2016

Related U.S. Application Data
(60) Provisional application No. 62/142,949, filed on Apr. 3, 2015.

(51) Int. Cl. A61K 31/65; A61K 9/50; A61K 31/66; A61K 9/00

(52) U.S. Cl. CPC A61K 31/65; A61K 9/0014; A61K 9/50; A61K 31/66

(57) ABSTRACT

A topical composition for treatment of a skin condition comprising a fluorescent, photosensitive, or oxygen sensitive active ingredient encapsulated in a plurality of tubular microparticles for pharmaceutical use in humans is described. Some embodiments comprise a topical composition for treatment of acne that comprises an encapsulation of a tetracycline class drug, such as crystalline minocycline, in a plurality of microparticles, wherein the microparticles comprise a divalent cation, such as Mg²⁺ or Zn²⁺. The tubular microparticles may comprise Mg₂CO₃. Benefits of various embodiments include the lack of visible fluorescence from a fluorescent active ingredient, reduced UV degradation of a photosensitive active ingredient, reduced UV degradation of an oxygen sensitive active ingredient, sun protection factor for the skin, and sustained delivery of a therapeutic dose.

### CROSS-REFERENCE TO RELATED APPLICATIONS

This application claims the benefit of priority to U.S. Provisional Patent Application No. 62/142,949, filed Apr. 3, 2015, which is incorporated herein by reference in its entirety.

### BACKGROUND OF THE INVENTION

This invention relates generally to a composition comprising a fluorescent, photosensitive, or oxygen-sensitive active ingredient encapsulated in a microparticle for pharmaceutical use in humans. More particularly, it relates to encapsulation of a fluorescent, photosensitive, or oxygen-sensitive active ingredient in a microparticle, such that light or oxygen is limited from penetrating to or emitting from the active ingredient and such that the active ingredient has an extended release period.

Autoimmune diseases such as psoriasis, eczema and scleroderma resulting in compromised barrier function of the skin can benefit from selected active ingredients that can be released sustainably without being degraded by sun or oxygen exposure. For example, cyclosporine, methotrexate, mycophenolate mofetil, and clobetasol propionate are active ingredients used in compositions prescribed for the treatment of autoimmune skin diseases. Some of these compositions are typically prescribed to be applied more than once daily due to the rapid degradation of the active ingredient following application to the skin and exposure to light (especially sunlight) and oxygen.

Skin photoaging as a result of chronic sun exposure has been addressed with numerous treatment options. Laser skin resurfacing provides a costly means to treating severe sun damage. Topical delivery of active ingredients provides the potential for a more economical solution for several skin conditions, including actinic keratosis. However, many topical compositions, such as many containing 5 fluorouracil (5FU), provide limited benefits due to degradation of the active ingredient following exposure to light and oxygen.

Acne is a medical condition that is believed to be caused by several factors including bacteria (e.g. propionibacterium acne, also known as p. acne), excessive production of sebum, enlargement of sebaceous glands due to androgen hormones, excessive flaking of skin cells, irritation and inflammation of skin hair follicles, and genetic factors. Common treatments include washing the affected area regularly with soap and water, topical application of an active ingredient, such as benzoyl peroxide, sulfur, retinoids, salicylic acid, azelaic acid, clindamycin, erythromycin, dapsone, or systemic active ingredients taken orally, such as antibiotics (minocycline, erythromycin or clindamycin) or isotretinoin (a retinoid). Each of these solutions addresses one or more of the causes of acne. However, each is also associated with side effects or is insufficient in many cases. Oral isotretinoin was a commonly prescribed acne treatment until its recent association with birth defects, liver damage, depression, and irritable bowel disease.

There are several active ingredients that have been used in topical compositions for the treatment of acne. Several of these effective active ingredients, such as minocycline, are fluorescent. Minocycline fluoresces when exposed to UV light, which can cause an unflattering appearance, particularly in the presence of a “black light” in an otherwise dark environment, such as at a nightclub. Such fluorescence can draw unwanted attention to a patient's acne condition. Patient concern about such fluorescence can impair compliance and commercial acceptability among patients for otherwise effective active ingredients and compositions.

In U.S. Pat. No. 8,258,327, Marto et al. disclose processes for the creation of crystalline forms of tetracycline class drugs, including minocycline base. In U.S. patent application Ser. No. 13/380,283, Heggie discloses a composition that uses crystalline forms of tetracycline class drugs, including minocycline base. These crystalline forms of tetracycline are described as more stable than amorphous forms of tetracycline, but are still susceptible to degradation by exposure to light and oxygen.

For many active ingredients, such as retinoids, topical application is preferred in comparison to oral delivery due to the focused local application and to the limited involvement of organs other than skin in the treatment.

For topical applications, sustained delivery of the active ingredient would be a desirable feature to allow a longer duration of delivery of the active ingredient. Many commonly used active ingredients degrade upon exposure to light. For this reason, a sustained delivery mechanism that limits or eliminates the exposure of the active ingredient to light would be desirable.

Some topically applied compositions include active materials that degrade when exposed to light or oxygen. Such compositions can require special packaging or special user instructions to minimize exposure of the active ingredient to light or oxygen. For this reason, a composition that limits or eliminates the exposure of the active ingredient to light or oxygen is desirable.

Many encapsulation mechanisms have been used topically to deliver drug compositions. However, many such encapsulations limit the amount of drug that can be delivered due to small ratios of the encapsulated active ingredient to the amount of encapsulant used.

Acne and many other skin conditions can be aggravated by sun exposure. A topical composition that limits the exposure of the patient's skin to sunlight is desirable.

U.S. Pat. No. 4,081,528 describes solutions comprising tetracycline, a soluble magnesium compound, and 2-pyrrolidone. This solution is described as having particular importance for veterinary, parenteral use. The concentration of the magnesium compound is said to be soluble in the tetracycline solution with a molar ratio of about 0.8 to 1.3 mole with the goal of creating a “clear stable solution.” Such a clear, stable solution does not protect the tetracycline from light-based degradation.

There is a need for a topical composition that comprises a photosensitive, oxygen-sensitive, or fluorescent active ingredient and whereby the composition limits the exposure of active ingredient to light or atmospheric oxygen and allows sufficient delivery of the active ingredient to the skin. Such a composition would desirably have one or more of the following attributes: notably reduces the fluorescence of an active ingredient relative to the active ingredient out of composition, limits degradation of active ingredients due to light or oxygen exposure, limits the amount of sunlight reaching the skin to which it was applied, and allows sustained delivery of an active ingredient to the skin.

### SUMMARY OF THE INVENTION

The present invention overcomes the limitations of the prior art by providing a composition for topical application comprising an active ingredient and a plurality of tubular microparticles which contain the active ingredient.

In some embodiments, the composition is used for the treatment or prophylaxis of a dermatological pathology and the active ingredient of the composition is fluorescent, photosensitive, or oxygen-sensitive.

In some embodiments, the composition is used for the treatment or prophylaxis of a cutaneous infection, acne, or cutaneous autoimmune disease and the active ingredient is fluorescent, while the composition is not fluorescent.

In some embodiments, the composition is used for the treatment or prophylaxis of a cutaneous infection, acne, or cutaneous autoimmune disease and the active ingredient is photosensitive, while the composition is not photosensitive or the rate of photo-degradation of a potency of the active ingredient is reduced by at least 10% or at least 50% relative to the active ingredient alone or relative to the composition prepared without the use of microparticles.

In some embodiments, the composition is used for the treatment or prophylaxis of a cutaneous infection, acne, or cutaneous autoimmune disease and the active ingredient is oxygen sensitive, while the composition is not oxygen sensitive or the rate of oxygen degradation of a potency of the active ingredient is reduced by at least 10% or at least 50% relative to the active ingredient alone or relative to the composition prepared without the use of microparticles.

In some embodiments, the composition is used for the treatment or prophylaxis of a cutaneous infection, acne, or cutaneous autoimmune disease and the active ingredient is fluorescent and photosensitive, while the composition is neither fluorescent nor photosensitive.

Some embodiments of the composition comprise a lipophilic delivery base or a hydrophilic delivery base.

Some embodiments of the composition comprise tubular microparticles with an internal volume in the range of 2.5 to 300,000 cubic micrometers or 10 to 1000 cubic micrometers.

In some embodiments of the composition, the ratio of mass of the active ingredient to mass of the plurality of tubular microparticles is 1:1,000,000 to 1:1,000 or 1:1,000 to 15:1.

In some embodiments of the composition, the concentration of the plurality of tubular microparticles is 0.0001 to 0.1 milligrams per milliliter, 0.1 to 100 milligrams per milliliter, or 100 to 10,000 milligrams per milliliter.

In some embodiments, a portion of the plurality of tubular microparticles are porous.

In some embodiments, a portion of the plurality of tubular microparticles comprise a compound with a divalent cation. The compound may be insoluble in the composition. The divalent cation may be a magnesium cation or a zinc cation.

In some embodiments, a portion of the plurality of tubular microparticles comprises magnesium carbonate (Mg₂CO₃).

In some embodiments, a portion of the plurality of tubular microparticles comprise magnesium carbonate and the active ingredient comprises minocycline, tetracycline, or a tetracycline derivative. In some embodiments the molar ratio of the active ingredient, for example tetracycline or a tetracycline derivative, to magnesium carbonate is in the range of 0.001 to 0.75.

In some embodiments, the active ingredient comprises one or more crystalline forms of tetracycline class drugs, such as those described in U.S. Pat. No. 8,258,327. Examples of crystalline forms of tetracycline class drugs include crystalline forms of minocycline, such as Form I, Form II, and Form III as described by U.S. Pat. No. 8,258,327.

The crystalline form of one or more tetracycline class drugs, such as one or more forms of crystalline minocycline base, may be embedded in a lipophilic embedding base and a hydrophilic delivery base.

In some embodiments, a portion of the plurality of tubular microparticles comprises a material that reacts with acids on the skin to degrade the strength and/or the integrity of the tubular microparticles.

In some embodiments, the composition is designed such that a portion of the microparticles fracture when rubbed on the skin during topical application of the composition.

In some embodiments, the composition further comprises a material with a melting temperature in the range of 20-40° C.

In some embodiments, the composition is not transparent or is optically scattering.

In some embodiments, the active ingredient comprises one or more of minocycline, tetracycline, bacitracin, neomycin, polymyxin, clobetasol propionate, methotrexate, tretinoin, sulfa antibiotics, ciprofloxacin, an ingredient that is activated by light, 5-aminolevulinic acid, and methyl aminolevulinate. In some embodiments, the composition comprises minocycline, tetracycline, or a tetracycline derivative in a concentration of 0.5% to 20% by weight or 2% to 10% by weight.

In some embodiments, the composition is used in the treatment of acne and may comprise one or more of the following: minocycline, a cycline-class antibiotic, a mycin-class antibiotic, anti-inflammatory, salicylic acid, benzoyl peroxide, botulinum toxin, vitamins, vitamin derivatives, minerals, peptides, and vitamin C.

In some embodiments, the composition is used in the treatment of a cutaneous infection or an autoimmune disease. In some embodiments, the autoimmune disease that is treated by the composition is psoriasis, eczema, or scleroderma. In some embodiments, an autoimmune disease is treated with a composition comprising one or more of cyclosporine, methotrexate, mycopholate mofetil, and clobetasol propionate.

In some embodiments, the composition is used in the treatment of a photodamaged skin or photo-induced disease. In some embodiments, the disease that is treated by the composition is actinic keratosis or basal cell carcinoma. In some embodiments, the skin is treated with a composition comprising 5-fluorouracil.

In some embodiments, the topical composition is applied no more frequently than once daily.

In some embodiments, the release rate of the active ingredient is 10% to 90% per hour.

In some embodiments, at least two tubular microparticles have a minimum cross-sectional dimension in the range of 1 to 50 micrometers or 1 to 10 micrometers.

In some embodiments, at least two tubular microparticles have a maximum cross-sectional dimension in the range of 10 to 500 micrometers or 50 to 200 micrometers.

In some embodiments, the ratio between the minimum and maximum cross-sectional dimension for at least two tubular microparticles is in the range of 1:1 to 1:200; 1:5 to 1:100; or 1:10 to 1:50.

In some embodiments, the active ingredient is fluorescent and photosensitive.

In some embodiments, the active ingredient is fluorescent, photosensitive, or oxygen-sensitive.

In some embodiments, the composition repels mosquitoes when topically applied.

In some embodiments, the composition has a sun protection factor of 4 to 100 or 15 to 60.

In some embodiments, the active ingredient is photosensitive, such as a retinoid or 5-fluorouracil, and reverses at least one effect of cutaneous photodamage, and the composition has a sun protection factor of at least 15.

In some embodiments, the composition is intended for the treatment of wrinkles or fine lines and may comprise a retinoid.

Methods of preparing the compositions described above are also presented.

### BRIEF DESCRIPTION OF THE DRAWINGS

FIG. 1a illustrates a composition comprising a plurality of tubular microparticles that each contains an active ingredient. FIG. 1b illustrates a composition comprising a topical base and a plurality of tubular microparticles that each contains an active ingredient.

FIG. 2a illustrates an example of an active ingredient mixed into an embedding base.

FIG. 2b illustrates an example of a composition comprising a topical base with a plurality of tubular microparticles that each contains a mixture as shown in FIG. 2a.

FIGS. 3a-3f illustrate multiple examples of embodiments of shapes of tubular microparticles. FIG. 3g is a scanning electron micrograph of an embodiment of a tubular microparticle.

FIGS. 4a and 4b are images of the fluorescence given off by different compositions in response to illumination with a UV fluorescent lamp. The images of FIGS. 4a and 4b were captured using a digital camera connected to a microscope. FIG. 4a shows an image of the fluorescence of minocycline hydrochloride (HCl). FIG. 4b shows an image of the fluorescence of minocycline contained in a plurality of Mg₂CO₃ tubular microparticles with a concentration of 1:100 of minocycline to Mg₂CO₃ tubular microparticles (i.e., 10 milligrams of minocycline HCl for every 1 gram of tubular microparticles) and illuminated and imaged under the same conditions as for FIG. 4a.

FIGS. 5a and 5b are images of the fluorescence given off by different compositions in response to illumination with a UV fluorescent lamp. The images of FIGS. 5a and 5b were captured using a digital camera connected to a microscope. FIG. 5a shows an image of the fluorescence of unencapsulated clindamycin. FIG. 5b shows an image of the fluorescence of clindamycin contained in a plurality of Mg₂CO₃ tubular microparticles with a concentration of 3:100 of clindamycin to Mg₂CO₃ tubular microparticles (30 milligrams of clindamycin for every 1 gram of tubular microparticles) and illuminated and imaged under the same conditions as for FIG. 5a.

FIG. 6 is a graph showing the mass of minocycline released into a phosphate buffered saline (PBS) solution as a function of time from a composition comprising a plurality of tubular microparticles that contain minocycline.

FIG. 7 shows the light spectrum measured by a spectrophotometer in transmission mode for a sample of Mg₂CO₃ tubular microparticles mixed into glycerin in a concentration of 2.5 milligrams per milliliter in a 4 cubic centimeter quartz cuvette. The transmission data are normalized relative to that of a sample of glycerin without tubular microparticles in the quartz cuvette.

### DEFINITIONS

Active ingredient means an agent that causes a desired clinically measurable response from a biological system. An active ingredient can, for example, be a drug, vitamin, or vitamin derivative.

Photosensitive describes an active ingredient that has a potency that degrades at a rate that is at least 50% larger when exposed to sunlight at its typical storage temperature and humidity or at its typical usage temperature and humidity than when in a dark room at the same temperature and humidity conditions.

A substance is considered to be fluorescent if, when illuminated with a 4-Watt Woods lamp held at a distance of 6 inches from the substance in an otherwise dark room, it fluoresces light with intensity sufficient to be visible to the naked eye. Light that is simply reflected from the Woods Lamp illumination is not considered fluorescence.

Fluorescence describes the light emitted by a fluorescent substance.

Oxygen-sensitive describes an active ingredient that has a potency that degrades at a rate that is at least 50% larger at its typical storage temperature or at its typical usage temperature when exposed to air, which consists essentially of approximately 20% oxygen and approximately 80% nitrogen, than when exposed to a nitrogen environment at the same temperature condition.

Tubular describes a 3-dimensional shape that has at least one 2-dimensional cross-section comprising a hollow closed loop of material and that has at least one opening with a cross-sectional area of at least 0.5 μm². A tubular microparticle may be made in a variety of shapes, including tube, bent, T-shaped, or bottle-shaped. A tubular microparticle may be open on only one end or may have two or more openings.

The maximum cross-sectional dimension of an object, such as a tubular microparticle, is the maximum linear distance between two points of the object.

The minimum cross-sectional dimension of an object, such as a tubular microparticle, is the minimum of all cross sectional dimensions for the object. A cross sectional dimension of an object is measured by first taking the projection of the 3-dimensional object onto a 2-dimensional plane and then projecting the 2-dimensional projection onto a 1-dimensional line in that plane. The cross-sectional dimension is the maximum distance between points of the 1-dimensional projected image on the line. For a cylindrical tubular microparticle with a diameter that is shorter than the tube length, the minimum cross-sectional dimension is the outer radius of the tubular microparticle.

A microparticle is defined as an object with a maximum cross-sectional dimension in the range of 5 to 500 micrometers, inclusive, and a minimum cross-sectional dimension in the range of 1 to 100 micrometers, inclusive.

A plurality of tubular microparticles is said to contain a portion of a substance if a portion the substance or all of the substance is included within the volumes of individual tubular microparticles within the plurality. The volume of a tubular microparticle is defined as the smallest closed volume that contains all of the line segments formed by joining all points of the tubular microparticle to all other points of the tubular microparticle.

### DETAILED DESCRIPTION OF THE PREFERRED EMBODIMENTS

Additional understanding of the invention, including particular aspects, embodiments, and advantages, will be apparent by referring to the detailed description below and to the drawings.

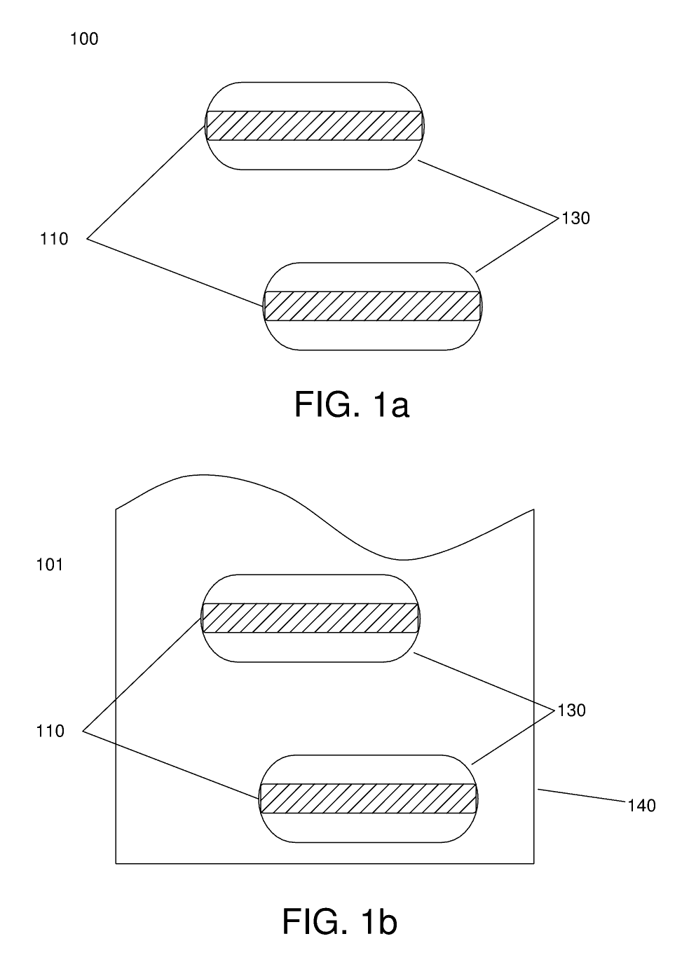

FIGS. 1a and 1b are illustrations of compositions according to embodiments of the invention. In FIG. 1a, a composition 100 comprises an active ingredient 110 and a plurality of tubular microparticles 130.

FIG. 1b illustrates a composition 101 that comprises an active ingredient 110 and a plurality of tubular microparticles 130 are distributed within a delivery base 140, which can be used to improve the delivery qualities for the composition 101 in comparison to composition 100 in certain applications.

The active ingredient 110 can be fluorescent, photosensitive, or oxygen sensitive. Each of the plurality of tubular microparticles 130 contains a portion of the active ingredient 110. Thus, the plurality of tubular microparticles 130 shields the contained portions of active ingredient 110 from exposure to light, from exposure to oxygen, or from emitting light that would be visible to a patient or others.

Note there may be tubular microparticles within the composition that do not contain any active ingredient 110 and there may be excess active ingredient 110 that is not contained by any tubular microparticles.

The active ingredient 110 provides key aspects of the clinical benefit derived from the composition. Examples of embodiments of the active ingredient 110 include drugs, vitamins, or vitamin derivatives that are fluorescent, such as cycline-class and mycin-class antibiotics. Examples of cycline-class antibiotics that are examples of active ingredients 100 include tetracycline class drugs such as minocycline HCl, doxycycline, oxytetracycline, and tetracycline. Examples of mycin-class antibiotics that are examples of active ingredients 100 include clindamycin and lincomycin. Other examples of embodiments of the active ingredient 110 include drugs, vitamins, or vitamin derivatives that are photosensitive, such as retinoids, 5-aminolevulinic acid, and vitamin C. Other examples of the active ingredient 110 include drugs, vitamins, or vitamin derivatives, such as botulism toxin, that would benefit from the sustained release profile enabled by encapsulation in a tubular microparticle.

The delivery base 140 is chosen based on the delivery characteristics needed for the composition. A composition 100 that is to be applied topically and needs to have only a short interaction with the skin could comprise a delivery base 140 that is hydrophilic to improve the tactile characteristics of the composition. For a composition 100 that is to be applied topically and needs an interaction that lasts for several minutes or hours, the composition could incorporate a lipophilic delivery base 140.

The tubular microparticles 130 are chosen to retain the active ingredient such that the active ingredient 110 is shielded from external light or oxygen. The tubular microparticles making up the plurality of tubular microparticles 130 are typically chosen with a minimum cross-sectional dimension in the range of 1 to 50 micrometers. In some applications, the minimum cross-sectional dimension of tubular microparticles making up the plurality of tubular microparticles 130 is in the range of 1 to 10 micrometers. The maximum cross-sectional dimension of the tubular microparticles making up the plurality tubular microparticles 130 is in the range of 10 to 500 micrometers. In some applications, the maximum cross-sectional dimension of the tubular microparticles making up the plurality tubular microparticles 130 is in the range of 50 to 200 micrometers or 10 to 50 micrometers. Preferably, the ratio between the minimum cross-sectional dimension and the maximum cross-sectional dimension is between 1:1 and 1:200 and for many applications, this ratio is in the range of 1:5 to 1:100 or 1:10 to 1:50. The thickness of the walls of the tubular microparticles is typically in the range of 0.25 to 10 micrometers. The cross sectional inner diameter is typically in the range of 0.5 to 40 micrometers or 1 to 5 micrometers depending on the desired characteristics, such as delivery method and delivery rate, for the composition.

One advantage of encapsulation by a plurality of tubular microparticles 130 with maximum cross-sectional dimension of between 10 and 100 micrometers is that penetration into a hair follicle or through a portion of damaged epithelium for sustained release can be achieved. Such tubular microparticles are small enough to penetrate into the hair follicle, too large to penetrate into an unbroken epithelium, and large enough to deliver therapeutic doses of drug over a period of 1 to 30 days.

The plurality of tubular microparticles 130 can comprise a divalent cation, such as Mg²⁺ or Zn²⁺.

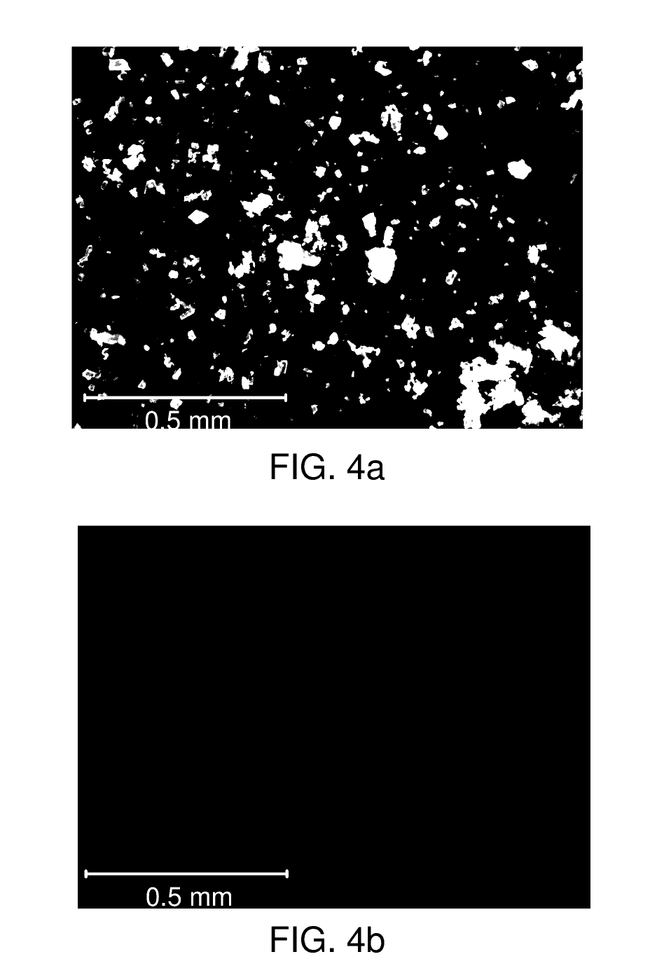

In one embodiment, the plurality of tubular microparticles 130 comprises Mg₂CO₃ and the active ingredient 110 is minocycline. As shown by comparison of FIG. 4a with FIG. 4b, the fluorescence of minocycline is suppressed in comparison to unencapsulated minocycline. The divalent cation Mg²⁺ suppresses the fluorescence of the minocycline when the minocycline is exposed to UV illumination.

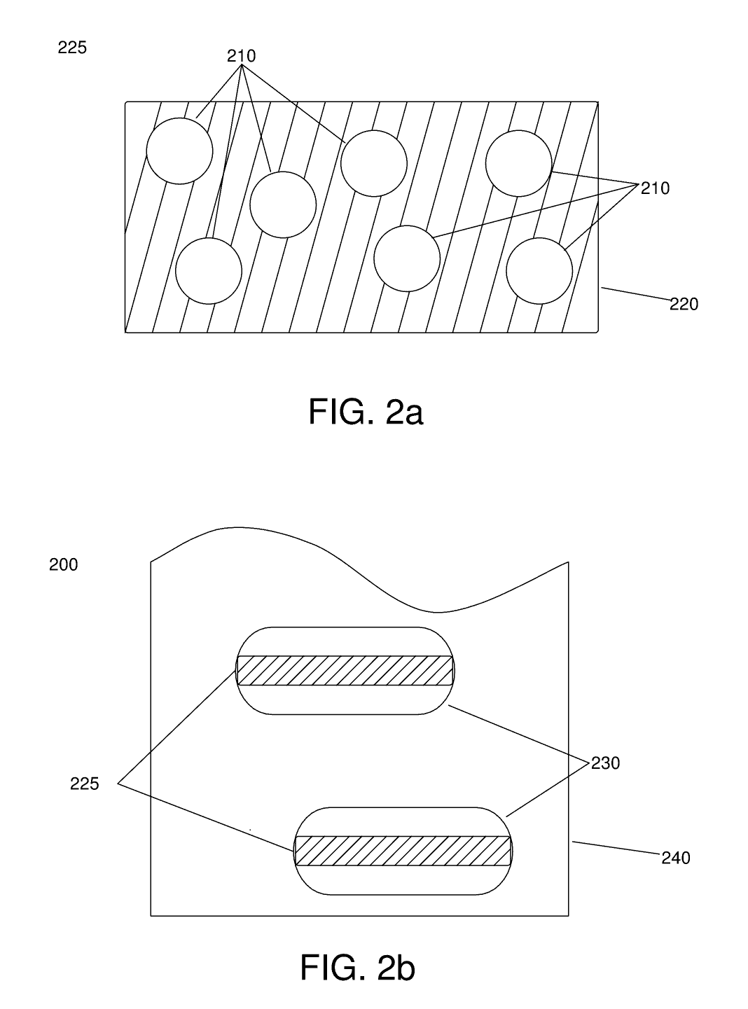

FIG. 2b illustrates a composition 200 that comprises an active ingredient 210, an embedding base 220, a plurality of tubular microparticles 230, and a delivery base 240. The active ingredient 210 and the embedding base 220 are mixed together as shown in FIG. 2a to form an active ingredient mixture 225, which is contained by each of the plurality of tubular microparticles 230. This plurality of tubular microparticles 230 is mixed into the delivery base 240.

The composition 200 comprises a plurality of tubular microparticles 230, an active ingredient 210, and a delivery base 240. The composition 200 further comprises an embedding base 220 in which the active ingredient is mixed, typically before the active ingredient is inserted into the plurality of tubular microparticles 230. This embedding base 220 allows the active ingredient 210 to have limited interaction with the delivery base 240, which allows more flexibility in the choice of delivery base 240.

The embedding base 220 can be either lipophilic or hydrophilic, depending on the desired characteristics of the delivery base 240 and active ingredient 210.

For example, a lipophilic embedding base 220 can be used with a hydrophilic delivery base 240 to minimize the dissolution of the embedding base 220 into the delivery base 240. Using a lipophilic embedding base 220 in a hydrophilic delivery base 240 can reduce the release rate of the active ingredient 210 from the plurality of tubular microparticles 230. Lipophilic examples of embodiments of the embedding base 220 are plasticized ointment, mineral oil derivatives, and polyethylene glycol.

The embedding base 220 can be hydrophilic, which can be particularly useful when combined with a lipophilic delivery base 240 to minimize the dissolution of the embedding base 220 into the delivery base 240. Hydrophilic examples of the embedding base 120 are creams, gels, and foams.

In many embodiments, the active ingredient 210 and embedding base 220 are either both lipophilic or both hydrophilic. One advantage of this approach is to minimize the amount of the active ingredient 210 that will precipitate.

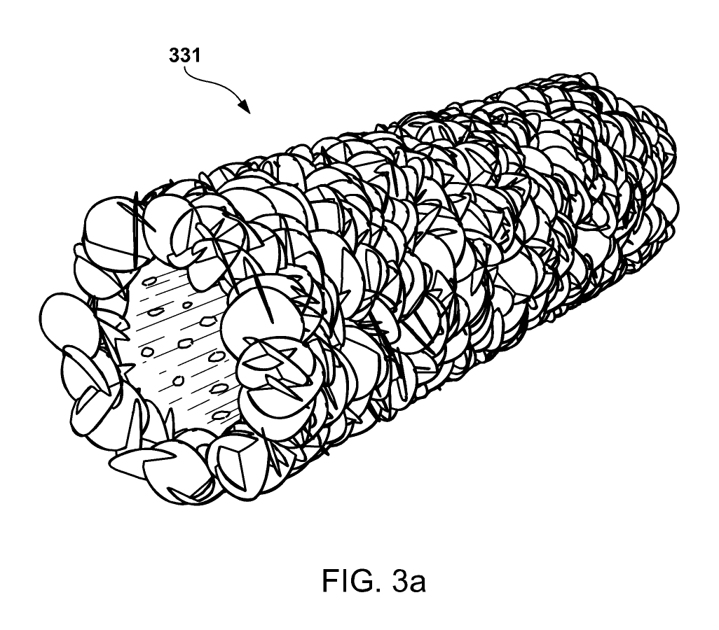

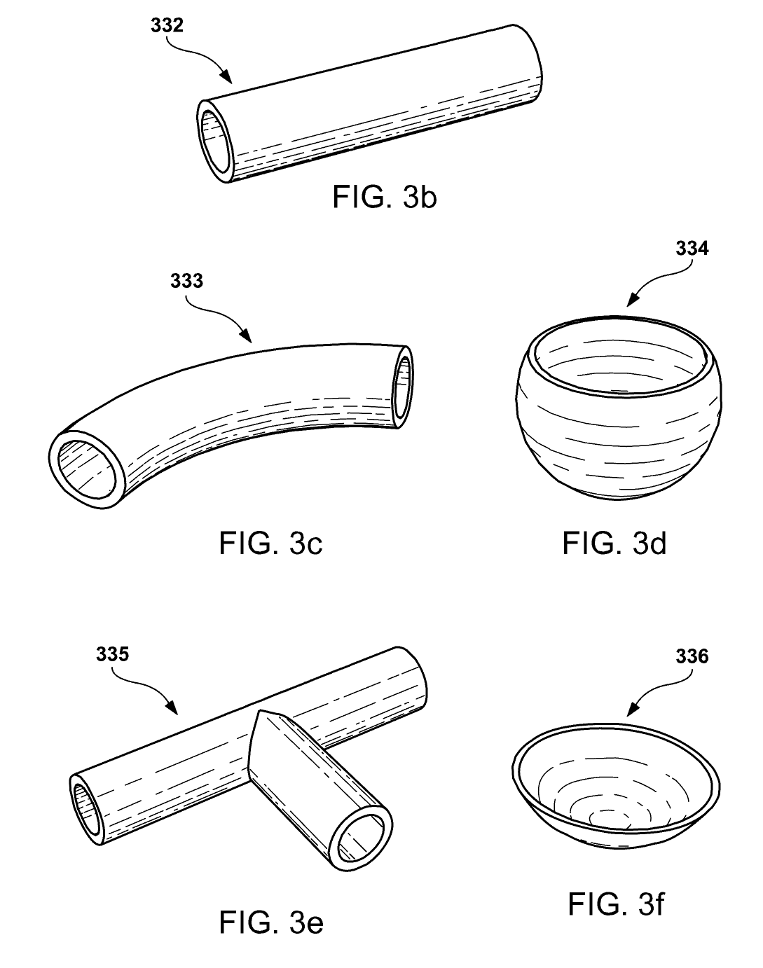

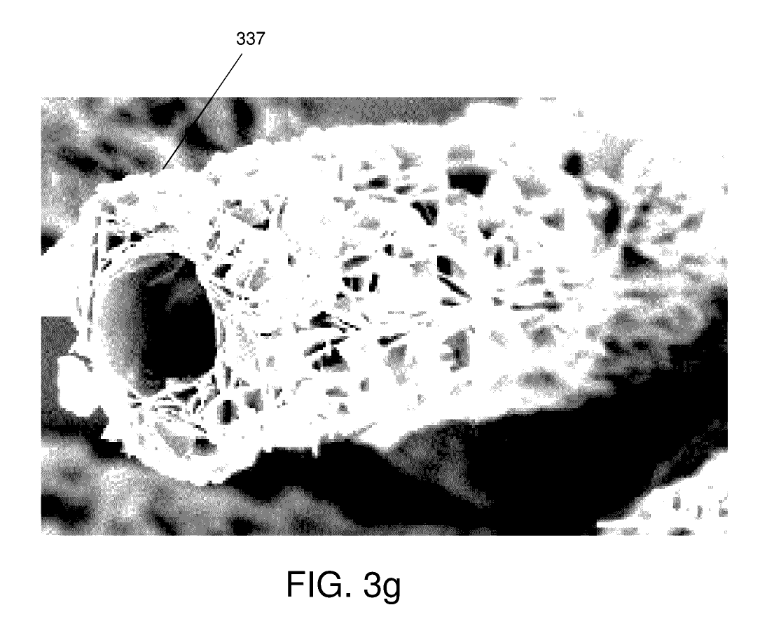

FIGS. 3a-3g show illustrative examples of tubular microparticles. Tubular microparticle shapes are shown in the perspective view drawings shown in FIGS. 3a-3f. FIG. 3a shows a porous hollow cylinder shaped tubular microparticle 331. Any of the shapes shown in FIGS. 3b-3f can be porous or non-porous. FIG. 3b shows a hollow cylinder shaped tubular microparticle 332. FIG. 3c shows an angled-tube shaped microparticle 333. A truncated spheroid shaped tubular microparticle 334 is shown in FIG. 3d. This shape is nearly spherical in shape with an opening in its surface. FIG. 3e shows a T-shaped microparticle 335. FIG. 3f shows a micro-bowl-shaped microparticle 336. The choices of shapes and dimensions of the tubular microparticles comprising the plurality of tubular microparticles will depend on a number of factors including the viscosity of the embedding agent, the surface tension between the embedding base 220 and the tubular microparticles in the plurality of tubular microparticles 230, the desired rate of release of the active ingredient 110, the amount of capillary action between the embedding base 220 and the tubular microparticles in the plurality of tubular microparticles 230, and the desired amount of shielding of the active ingredient 110 from ambient light. FIG. 3g is an image of a porous hollow cylinder shaped magnesium carbonate tubular microparticle 337. The image was created by a scanning electron microscope.

The concentration of tubular microparticles in the plurality of tubular microparticles 130, the size of each tubular microparticle in the plurality of tubular microparticles 130, and the size of the opening or openings in each tubular microparticle in the plurality of the tubular microparticles 130 is typically selected based on the desired release rate of the active ingredient and the amount of optical shielding or oxygen shielding that is needed.

The size of openings of the tubular microparticles also has an effect on the ability to fill the tubular microparticles in the plurality of tubular microparticles 130. Capillary action can be used to fill the plurality of tubular microparticles 130 with active ingredient 110 or active ingredient mixture 225. The ability to fill the plurality of tubular microparticles 130 can be improved by appropriate selection of the material for the plurality of tubular microparticles 130, the diameter for each of the plurality of tubular microparticles 130, and the diameter of the opening or openings in each of the tubular microparticles in the plurality of tubular microparticles 130. The tubular microparticles in the plurality of tubular microparticles 130 can be all of the same dimensions or can differ in one or more dimensions, such as the area of one or more openings or minimum or maximum cross-sectional diameter, such as to deliver the active ingredient at different rates and over different durations of action.

FIG. 4b shows an image of the fluorescence given off by a composition in response to illumination with a UV fluorescent lamp. The composition includes minocycline contained in a plurality of Mg₂CO₃ tubular microparticles with a concentration of 1:100 of minocycline to Mg₂CO₃ tubular microparticles (10 milligrams of minocycline per gram of Mg₂CO₃ tubular microparticles). FIG. 4a shows an image of the fluorescence given off by a composition of minocycline without a plurality of tubular microparticles.

The composition shown in FIG. 4b was made by dissolving minocycline HCl in methyl alcohol at a concentration of 10 mg/ml. A plurality of tubular microparticles comprising Mg₂CO₃ was added to the solution. Microscope slides were prepared with minocycline HCl (shown in FIG. 4a) and the prepared composition with a plurality of tubular microparticles described in this paragraph (shown in FIG. 4b). These two microscope slides were then inspected under fluorescence microscopy to produce the images shown in FIGS. 4a and 4b.

The control composition imaged in FIG. 4a showed fluorescence emission at approximately 670 nm with a 450 nm excitation wavelength. No fluorescence was observable in the composition imaged in FIG. 4b, even under microscope magnification at 10 times magnification. Fluorescence can be an indicator of photo-initiated degradation of some antibiotics, including minocycline.

Comparison of the fluorescence microscopy images shown in FIGS. 4a and 4b thus demonstrates that fluorescence from an active ingredient 110 that is contained in a plurality of tubular microparticles 130 can be suppressed relative to the active ingredient 110 without tubular microparticles.

The amount of fluorescence emitted by the active ingredient 110 under selected illumination conditions can be adjusted to yield different percentages of fluorescence relative to the unencapsulated active ingredient. The exact concentration active ingredient 110 relative to the plurality of tubular microparticles 130 can be selected based on the desired pharmaceutical or cosmetic characteristics of the composition. The percentage of fluorescent power emitted is desirably adjusted to be 0.001% to 20% of the fluorescent power emitted by the unencapsulated active ingredient 110. Alternately, the percentage of fluorescent power emitted is desirably adjusted to be 0.01% to 5% of the fluorescent power emitted by the unencapsulated active ingredient 110. Alternately, the percentage of fluorescent power emitted is desirably adjusted to be 0.1% to 1% of the fluorescent power emitted by the unencapsulated active ingredient 110. Alternately, the percentage of fluorescent power emitted is desirably adjusted to be less than 1% of the fluorescent power emitted by the unencapsulated active ingredient 110. The percentage of fluorescent power emitted can be measured as the average power emitted from a sample from a predefined area under predefined illumination conditions.

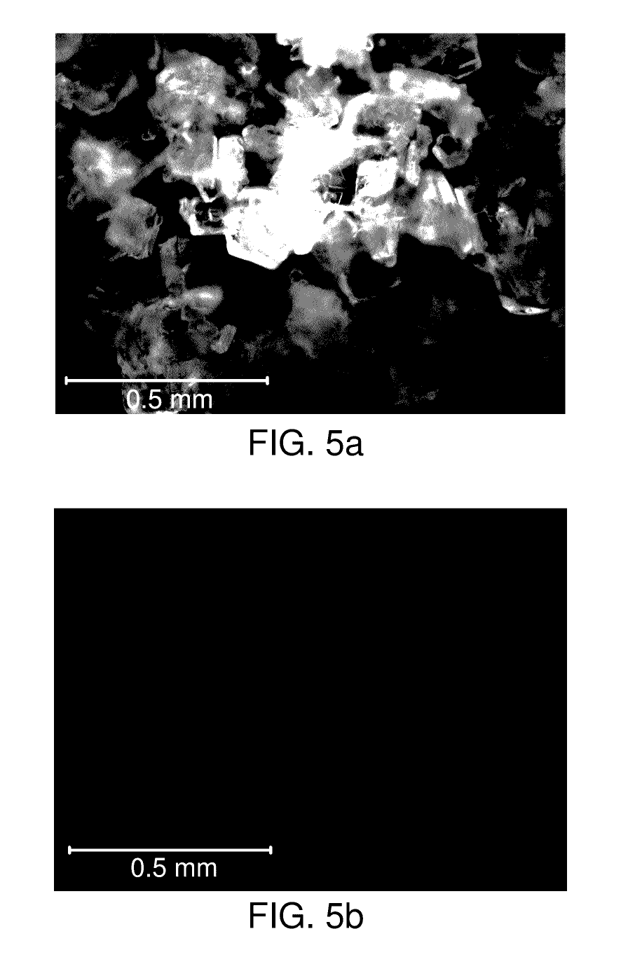

FIG. 5b shows an image of the fluorescence given off by a composition in response to illumination with a UV fluorescent lamp. The composition includes an active ingredient 110 (clindamycin) contained in a plurality of Mg₂CO₃ tubular microparticles with a concentration of 3:100 of clindamycin to Mg₂CO₃ tubular microparticles (30 milligrams of clindamycin per gram of Mg₂CO₃ tubular microparticles). FIG. 5a shows an image of the fluorescence given off by a composition of clindamycin without a plurality of tubular microparticles.

Comparison of the fluorescence microscopy images shown in FIGS. 5a and 5b demonstrates that fluorescence from an active ingredient 110 that is contained in a plurality of tubular microparticles 130 can be suppressed relative to the active ingredient 110 without tubular microparticles.

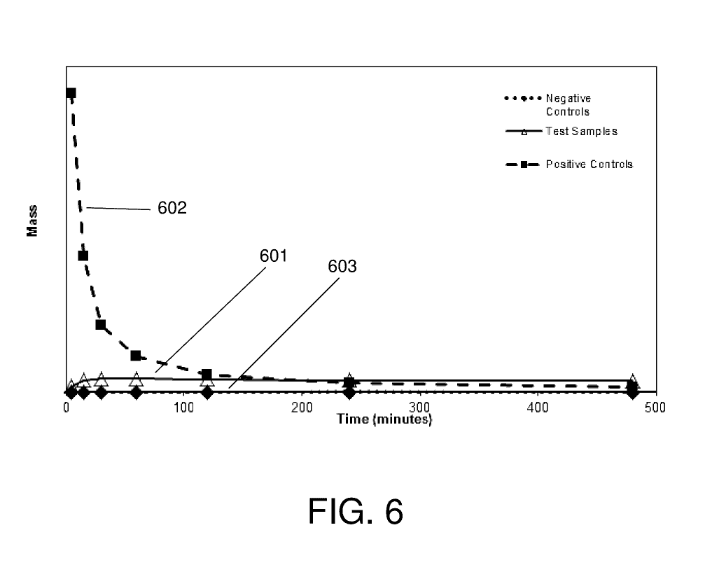

The graph shown in FIG. 6 describes sustained release data for three compositions as measured by sampling, over a period of 3 hours, the supernatant from three glass flasks that each held one of three compositions in phosphate buffered saline (PBS).

The first curve 601 corresponds to the data for the composition comprising a minocycline active ingredient contained in a plurality of tubular microparticles, which consisted essentially of magnesium carbonate. The concentration of active ingredient to tubular microparticles was approximately 1:11 (approximately 91 milligrams per gram).

The second curve 602 corresponds to the data for the composition comprising a minocycline active ingredient without a plurality of tubular microparticles.

The compositions corresponding to the first curve 601 and the second curve 602 were incubated in PBS for sampling. The amount of active ingredient (minocycline) in each sample was evaluated using a high performance liquid chromatography machine to produce the data plotted in FIG. 6.

The first curve 601 indicates that the composition comprising a plurality of tubular microparticles that contain a portion of the active ingredient had a sustained release rate for which the peak of the sustained release period was longer than 60 minutes and was longer than 30 minutes. Comparison of the first curve 601 and the second curve 602 show that the release rate of the active ingredient was reduced by the addition of a plurality of tubular microparticles. The plurality of tubular microparticles provided significant internal volume in which to contain a portion of the active ingredient and to reduce the initial release rate of active ingredient by a factor of 50% to 100%, by a factor of 90% to 100%, and by a factor of 98% to 100%.

The third curve 603 corresponds to the release rate of a topical composition consisting only of the topical base used for the samples described by the first curve 601 and the second curve 602. The sample corresponding to the third curve 603 served as the negative control for the experiment.

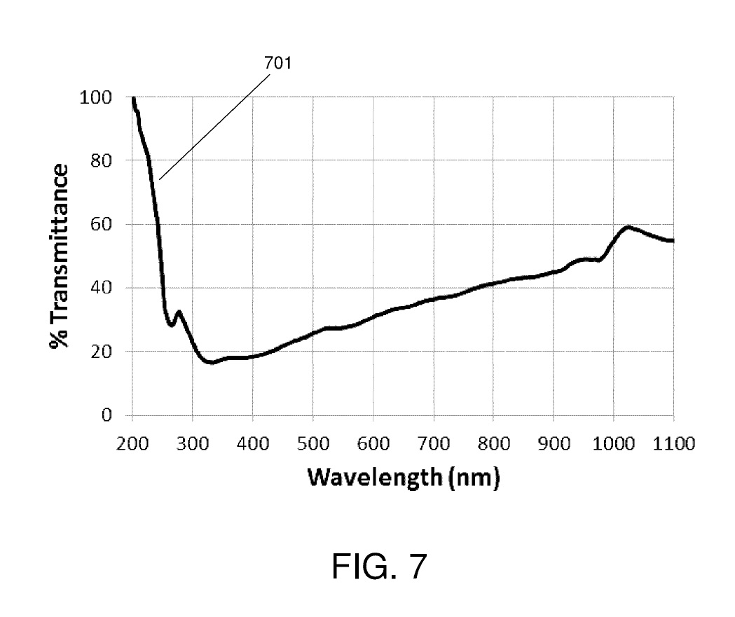

FIG. 7 shows the transmission light spectrum 701 measured by a photospectrometer in transmission mode for a sample comprising a plurality of tubular microparticles mixed into glycerin in a glass cuvette. The transmission data are normalized relative to a sample of glycerin in the glass cuvette. As shown in the data, the plurality of tubular microparticles provides an optical barrier that can shield harmful UV light from passing directly through it, such as ultraviolet-A (i.e., 315-400 nm) or ultraviolet B (i.e., 280-315 nm). This is useful in applications that benefit from topically applied compositions with sun protection. The sun protection factor (SPF) of such compositions can be adjusted based on the characteristics of the plurality of tubular microparticles 130 and the active ingredient 110 in the composition.

Additional data was collected with a high performance liquid chromatography (HPLC) machine for two samples. The first sample was minocycline HCl and the second sample comprised a plurality of microparticles that contained minocycline HCl. HPLC measurement can be used to detect photodegradation of a potency of minocycline by comparing the peaks for a photodegraded minocycline (epimerized minocycline) to undegraded minocycline. Following exposure of both samples to ultraviolet illumination for 10 hours, an HPLC machine was used to assess the level of degradation of the minocycline active ingredient in both samples. The minocycline active ingredient in the first sample degraded by 4.3% and the minocycline active ingredient in the second sample degraded by 99.3%. This indicates that the rate of degradation of the potency of the active ingredient is at least 50% lower than the degradation of the potency of the active ingredient without the plurality of tubular microparticles (data not shown).

### Example 1

In one embodiment of a composition according to the invention, the active ingredient is minocycline HCl (Sigma Aldrich, St. Louis, Mo.), the embedding base is plasticized ointment (Spectrum Chemical, Gardena, Calif.), the plurality of tubular microparticles consists of hollow cylinder-shaped tubular microparticles with an inner diameter of about 1 to about 5 micrometers and a length of about 10 to about 50 micrometers that consist essentially of magnesium carbonate (Nittetsu Mining Co., Tokyo, Japan). The minimum cross sectional dimension of the plurality of tubular microparticles is in the range of about 1 to about 5 micrometers and the maximum cross-sectional dimension of the plurality of tubular microparticles is in the range of about 10 to about 50 micrometers.

In this embodiment, the following sequence of steps is performed to prepare the composition: A fluorescent and photosensitive active ingredient, such as minocycline HCl is dissolved in a solvent and mixed in an evaporation flask until the particles are dissolved. A plurality of tubular microparticles comprising magnesium carbonate is mixed into the active ingredient mixture using a vortex mixer until the combination is homogenous in appearance. The flask containing the combination is set into a heated water bath at 40 degrees Celsius in a rotary evaporator while the pressure inside the flask is reduced to approximately 0 mBar over one minute. The pressure in the flask is maintained at approximately 0 mBar for 10 minutes while the flask is rotated within in the heated water bath of the rotary evaporator. Plasticized ointment base is added to the flask and rotated in the rotary evaporator at 50 degrees Celsius at approximately 0 mBar until the liquid has evaporated from the flask. The resulting powder material is collected from the flask and transferred to a container that shields its contents from excessive light exposure. The container is sealed to limit exposure to light and oxygen.

The lipophilic embedding base combined with the hydrophilic active ingredient and the plurality of hydrophilic tubular microparticles create surface tension forces that help to keep a portion of the active ingredient contained within the plurality of tubular microparticles.

This composition includes a lipophilic embedding base and a topical base. The topical base is a hydrophilic gel. Because the topical base is hydrophilic, it will dry quickly following application to the skin. This can be desirable in many applications.

Components of various embodiments include minocycline HCl (Sigma Aldrich, St. Louis, Mo.), plasticized ointment base (Spectrum Chemical, New Brunswick, N.J.), magnesium carbonate microtubes (Nittetsu Mining Co., Ltd., Tokyo, Japan), cyclopentasiloxane (Dow Corning Corp, Midland, Mich.), PEG (Dow Corning Corp, Midland, Mich.), PPG-18 (Dow Corning Corp, Midland, Mich.), 18 Dimethicone (Dow Corning Corp, Midland, Mich.), cyclomethicone (Dow Corning Corp, Midland, Mich.), dimethicone crosspolymer (Dow Corning Corp, Midland, Mich.), 0.75% solution of polyox in water (Amerchol Corporation, Edison, N.J.), and sodium chloride (Fisher Scientific Company L.L.C., Fair Lawn, N.J.).

### Example 2

In one embodiment of a hydrophilic composition according to the invention, the active ingredient is minocycline phosphate; the delivery base is a hydrophilic, water-based solution comprising cetylstearyl alcohol, Cremaphor A 6, Cremaphor A 25, liquid paraffin, and one or more parabene(s); the embedding base is propylene glycol; and the plurality of tubular microparticles consists of hollow cylinder-shaped tubular microparticles with an inner diameter of about 1 to about 5 micrometers and a length of about 10 to about 50 micrometers that consist essentially of magnesium carbonate. The minimum cross sectional dimension of the plurality of tubular microparticles is in the range of about 1 to about 5 micrometers and the maximum cross-sectional dimension of the plurality of tubular microparticles is in the range of about 10 to about 50 micrometers. Additional details about the complete composition for this example are given in Table 1.

To make the composition, the following steps can be performed. The following ingredients are mixed together: cetylstearyl alcohol, Cremaphor A 6, Cremaphor A 25, liquid paraffin, and one or more parabene(s). This mixture is heated to 80° C. Water at 80° C. is added to the mixture while stirring rapidly. In a separate container, the following ingredients are mixed together to form a second mixture: propylene glycol and minocycline phosphate. This second mixture is heated until the minocycline phosphate is dissolved in the propylene glycol. To the second mixture is added the magnesium carbonate microtubular particles while stirring. This second mixture is then mixed with the first mixture and stirred as the combined mixture is cooled to room temperature.

In this composition, cetylstearyl alcohol, Cremaphor A 6, Cremaphor A 25, and liquid paraffin act as thickening agents and parabene(s) acts as a preservative.

TABLE 1
Composition ingredients for composition of Example 2

| Concentration | |
|---|---|
| (by mass) | Ingredient |
| 7% | cetylstearyl alcohol |
| 1.5% | cremaphor A 6 |
| 1.5% | cremaphor A 25 |
| 12% | liquid paraffin |
| 0.1% | parabene(s) |
| 67.9% | water |
| 8% | propylene glycol |
| 1% | minocycline phosphate |
| 1% | magnesium carbonate tubular microparticles |

### Example 3

In one embodiment of a composition according to the invention, the active ingredient is minocycline phosphate; the delivery base is a hydrophilic, water-based solution comprising Lutrol E 400, propylene glycol, Lutrol F 127, and water; and the plurality of tubular microparticles consists of hollow cylinder shaped tubular microparticles with an inner diameter of about 1 to about 5 micrometers and a length of about 10 to about 50 micrometers that consist essentially of magnesium carbonate. The minimum cross sectional dimension of the plurality of tubular microparticles is in the range of about 1 to about 5 micrometers and the maximum cross-sectional dimension of the plurality of tubular microparticles is in the range of about 10 to about 50 micrometers. Additional details about the complete composition for this example are given in Table 2.

To make the composition, the following steps can be performed. The following ingredients are mixed together: Lutrol E 400 and propylene glycol. This mixture is heated to 70° C. Lutrol F 127 at 70° C. is added to the mixture until the first mixture dissolves in the Lutrol F 127. In a separate container, the following ingredients are mixed together to form a second mixture: water and minocycline phosphate. This second mixture is heated until the minocycline phosphate is dissolved in the water. To the second mixture is added the magnesium carbonate microtubular particles while stirring. This second mixture is then mixed with the first mixture and stirred as the combined mixture is cooled to room temperature.

TABLE 2
Composition ingredients for composition of Example 3

| Concentration | |
|---|---|
| (by mass) | Ingredient |
| 20% | Lutrol E 400 |
| 20% | propylene glycol |
| 20% | Lutrol F 127 |
| 38% | water |
| 1% | minocycline phosphate |
| 1% | magnesium carbonate tubular microparticles |

### Example 4

In an embodiment according to the invention, the active ingredient comprises one or more of the following: vitamins, vitamin derivatives, minerals, and peptides. Although there are some evidence that topical application of vitamins, vitamin derivatives, minerals, and peptides could aid in the protection and in some cases reversal of damaged skin in the case of long term and persistent use, benefits of such treatments are inconsistent and limited. A major factor in the unpredictability of such treatment is due to degradation of the active ingredients prior to or after application to the skin. Degradation could be caused by oxygen or direct sun exposure due to consumer lifestyle and behavior. Compositions according to this invention can address such limitations.

### Example 5

In some applications, it is beneficial to use an ointment delivery base to maintain moisture within the skin. In such cases, the use of a lipophilic embedding agent, a hydrophilic encapsulant, and a lipophilic delivery base can beneficially be combined in a composition.

### Example 6

In one embodiment of a composition according to the invention, the active ingredient is crystalline minocycline as described in U.S. Pat. No. 8,258,327, which is herein incorporated by reference in its entirety. The active ingredient used in the present invention can comprise one of the three examples of crystalline minocycline that are described in U.S. Pat. No. 8,258,327. These three examples are designated Form I, Form II, and Form III in U.S. Pat. No. 8,258,327 and that designation will be used herein.

In this embodiment, the following sequence of steps is performed to prepare the composition: (1) Mix a solution of 20 milligrams of crystalline minocycline to 1 milliliter of appropriate solvent until the crystalline minocycline is dissolved in the solvent. For crystalline minocycline of Form I, methyl tertiary butyl ether (MTBE) can be used. For Form II, ethyl acetate can be used. For Form III, ethanol can be used.

(2) Mix the following ingredients in a 2 ml polypropylene centrifuge tube (Thermo Fisher Scientific, Waltham, Mass.): (a) 50 mg of the plurality of tubular microparticles, (b) 0.25 ml of the crystalline minocycline solution prepared in step 1, and (c) 0.10 milliliters of a solvent (e.g., methanol, ethanol, or isopropanol). The solvent is selected to wet the microcarrier to enhance the embedding of the minocycline into the plurality of microtubular particles. Alternative solvents or solutions can be used for this purpose.

(3) Mix in a vortex mixer, such as the SI-0236 VORTEX-GENIE 2 mixer (Scientific Industries, Bohemia, N.Y.) at 600 revolutions per minute for 30 seconds.

(4) Evaporate substantially all of the solvent using a centrifugal vacuum concentrator (e.g., SAVANT SPEEDVAC centrifugal vacuum concentrator from Thermo Fisher Scientific, Waltham, Mass.). Samples can be heated to approximately 43° C. under vacuum while spinning in the centrifuge until evaporated (approximately 45 minutes). To reduce degradation of the sample due to light exposure, a preferred embodiment keeps samples in a dark environment during the drying process. The temperature to which the sample is heated can be from 10 to 50° C. or can vary within this range. Higher temperatures in this range (e.g., 35-50° C.) are typically preferred because they make the process proceed more quickly.

Steps (1) to (4) produce approximately 55 mg of the mixture of crystalline minocycline in the plurality of tubular microparticles. Larger amounts can be scaled from the proportions presented. Alternate ratios of the mass of crystalline minocycline to the mass of the plurality of tubular microparticles depend on the desired application. Ratios from 0.01 to 10 or ratios from 0.05 to 0.3 are particularly useful.

(5) The result of steps (1) to (4) can be mixed into a delivery base with desired characteristics such as touch, feel, and smell. This delivery base is preferably hydrophilic to better retain the minocycline within the plurality of tubular microparticles. For example, the external base could be formed from a mixture of water, cetylstearyl alcohol, cremaphor A 6, cremaphor A 25, liquid paraffin, and one or more parabenes. These can be mixed to create the desired characteristics for the topical application as well known to those skilled in the art.

In some compositions, the concentration of minocycline can be in the range of 0.1% to 5.0%.

In some embodiments, the composition may be designed such that rubbing the composition into the skin breaks a proportion, a majority, or substantially all of the plurality of microcarriers. Breaking one or more of the plurality of microcarriers allows a higher release rate for the active ingredient.

In some embodiments, the acid from the skin (or other usage environment condition) can break down the plurality tubular microparticles such that the release rate of the active ingredient is increased relative to the release rate into the delivery base prior to application on the skin. Particularly, the material that makes up the tubular microparticles can be selected such that the material can react with acids on the skin to degrade the strength and/or integrity of tubular microparticles. One example of such material is magnesium carbonate. H. Lambers, et al describe in an article (International Journal of Cosmetic Science; 2006 October; 28(5): 359-70) that the average pH of the skin is naturally approximately 4.7. At a pH of approximately 5.0, magnesium carbonate tubes breakdown and lose much of their mass within the period of 0.5 to 5 hours.

### Example 7

In one embodiment of a composition according to the invention, a variation of Example 6 can be used to enhance the amount of active ingredient that is contained by the plurality of tubular microparticles.

In this process, the steps are the same as in Example 6 with the exception that steps 1 and 2 are replaced by the following:

(1a) Mix a solution of 20 milligrams of crystalline minocycline to 1 milliliter of appropriate wetting solvent until the crystalline minocycline is dissolved in the wetting solvent. For crystalline minocycline of Form I, methyl tertiary butyl ether (MTBE) can be used. For Form II, ethyl acetate can be used. For Form III, ethanol can be used. Crystalline minocycline may beneficially penetrate bacteria associated with acne more effectively than amorphous forms of minocycline. This may allow lower dosages of minocycline to be required for an effective treatment if minocycline is used in the crystalline form.

(1b) Form an emulsion of the minocycline solution and an oil that dissolves in the selected wetting solvent from step (1a).

(2a) Mix the following ingredients in a 2 ml polypropylene centrifuge tube (Thermo Fisher Scientific, Waltham, Mass.): (a) 50 mg of the plurality of tubular microparticles and (b) 0.10 milliliters of a solvent (e.g., methanol, ethanol, or isopropanol). In this step, the solvent wets and fills the plurality of microtubular particles.

(2b) add to the centrifuge tube 0.25 ml of the crystalline minocycline solution prepared in step 1.

If the components of the emulsion are selected such that the emulsion has a lower boiling temperature than the wetting solvent introduced in step 1a, then the negative pressure created within the plurality of microtubular particles due to evaporation of the wetting solvent will help to draw the active ingredient into the plurality of tubular microparticles during the evaporation of step 4. This process is designated evaporative capillary action.

### Example 8

In one embodiment of a composition according to the invention, a variation of Example 7 can be used.

For this example, a suspension is used in place of an emulsion. To form the suspension, crystalline minocycline can be mixed with a suspension medium while the mixture heated. Preferably the suspension medium has a melting temperature in the range of 20° C. to 40° C. Examples of materials that could be used for the suspension medium are palm oil, which has a melting temperature of 35° C., red palm oil, which has a melting temperature of 24° C., and squalene, which has a melting temperature of 34° C. The suspension typically remains heated 1 to 10 degrees above its melting temperature during steps 2b and 3 to enable the suspended minocycline to move more easily into the plurality of microtubular particles.

One advantage of selecting a suspension medium with a melting temperature in the range of 20° C. to 40° C. is that this allows the emulsion to flow more easily when applied topically to the skin. For a person in a room temperature environment, the skin surface typically has a temperature of 32° C. to 35° C. With rubbing, as might be done with topical application, temperatures on the surface of the skin can rise to well over 40° C.

### Example 9

In other embodiments of compositions according to the invention, variations of Examples 6, 7, and 8 can be created in which crystalline minocycline is replaced by minocycline hydrochloride or other salt forms of minocycline. In such cases, an appropriate solvent for dissolving the minocycline could be propylene glycol.

In the embodiments described in Examples 6, 7, and 8, the embedding base is typically lipophilic while the delivery base is typically hydrophilic. If a salt form of minocycline is used, the embedding base is typically hydrophilic while the delivery base is typically lipophilic, such as a topical emollient (e.g., AQUABASE, Perrigo Company, Allegan, Mich.). The embedding base and delivery base are selected to have a different hydrophobicity in order to better isolate the active ingredient within the plurality of tubular microparticles.

### Example 10

In other embodiments of compositions according to the invention, variations of Examples 6, 7, and 8 can be created in which crystalline minocycline is replaced by crystalline forms of other tetracycline-class drugs or other lipophilic drugs.

We claim:

1. A topical composition for the treatment or prophylaxis of a dermatological pathology, comprising
an antibiotic active ingredient, and
a plurality of tubular microparticles comprising an enclosed volume,
wherein a portion of the antibiotic active ingredient is included within said enclosed volume of said tubular microparticles, and
wherein at least two tubular microparticles of the plurality of microparticles have a minimum cross-sectional dimension in the range of 1 to 50 micrometers.

2. The topical composition of claim 1, wherein
the antibiotic active ingredient is photosensitive, and
wherein a rate of degradation of a potency of the antibiotic active ingredient when exposed to an ultraviolet illumination is at least 10% lower than the degradation of the potency of the antibiotic active ingredient when exposed to the ultraviolet illumination for the topical composition prepared without the plurality of tubular microparticles.

3. The topical composition of claim 1, further comprising
at least two inactive ingredients,
wherein the amount of fluorescence from the topical composition when illuminated with UV illumination is at least 80% lower than the amount of fluorescence for a comparison mixture consisting of the at least two inactive ingredients and the fluorescent antibiotic active ingredient mixed in the same relative proportions as in the topical composition.

4. The topical composition of claim 1, wherein
the antibiotic active ingredient is fluorescent and photosensitive, and
the topical composition is neither fluorescent nor photosensitive.

5. The topical composition of claim 1, wherein
the antibiotic active ingredient is oxygen sensitive, and
the topical composition is not oxygen sensitive.

6. The topical composition of claim 1, wherein
a portion of the plurality of microparticles comprise microparticles with an internal volume in the range of 10 cubic micrometers to 1000 cubic micrometers.

7. The topical composition of claim 1, wherein
the ratio of mass of the antibiotic active ingredient to mass of the plurality of tubular microparticles is in the range of 1:1000 to 15:1.

8. The topical composition of claim 1, wherein
a portion of the plurality of tubular microparticles comprise a compound with a divalent cation.

9. The topical composition of claim 8, wherein
the divalent cation is chosen from the list consisting of a magnesium cation and a zinc cation.

10. The topical composition of claim 9, wherein
a portion of the plurality of tubular microparticles comprise magnesium carbonate.

11. The topical composition of claim 10,
wherein the antibiotic active ingredient comprises a cycline-class drug, and
wherein the molar ratio of the cycline-class drug to magnesium carbonate in the composition is in the range of 0.001 to 0.75.

12. The topical composition of claim 1, wherein
a portion of the plurality of tubular microparticles react with acids on the skin to degrade one or more of the strength and the integrity of the portion of the plurality of tubular microparticles.

13. The topical composition of claim 1, wherein
the topical composition comprises an ingredient that is activated by light.

14. The topical composition of claim 1, wherein
the antibiotic active ingredient comprises an ingredient chosen from the list consisting of a cycline-class drug and a mycin-class drug.

15. The topical composition of claim 1, wherein
the antibiotic active ingredient is minocycline or doxycycline.

16. The topical composition of claim 1, further comprising
a material with a melting temperature in the range of 20 to 40 degrees Celsius.

17. The topical composition of claim 1, wherein
the topical composition releases active the antibiotic active ingredient in the application environment at a rate in the range of 10% per hour to 90% per hour.

18. The topical composition of claim 1, wherein
at least two tubular microparticles of the plurality of microparticles have a maximum cross-sectional dimension in the range of 10 to 500 micrometers, inclusive.

19. The topical composition of claim 1, wherein
the topical composition has a sun protection factor and the sun protection factor is within the range of 4 to 100, inclusive.

20. A method for treatment or prophylaxis of a dermatological pathology comprising the steps of
applying the composition of claim 1 to a person's skin, and
repeating the applying step daily for a period of at least 6 weeks.
